(19) United States
(12) Patent Application Publication
(10) Pub. No.: US 2025/0241829 A1
(43) Pub. Date: Jul. 31, 2025

(54) Ceramic Inkjet Ink Compositions for Printing on Dental Restorations

(71) Applicant: Zima International, Inc. d/b/a Dandy, New York, NY (US)

(72) Inventors: Kenn Butler, New York, NY (US); Tri Tuladhar, Cambridge (GB); Kevin Myers, New York, NY (US)

(21) Appl. No.: 19/043,001

(22) Filed: Jan. 31, 2025

Related U.S. Application Data
(60) Provisional application No. 63/627,670, filed on Jan. 31, 2024.

(51) Int. Cl. A61K 6/17; A61K 6/20; A61K 6/811; A61K 6/813; A61K 6/816

(52) U.S. Cl. CPC A61K 6/17; A61K 6/20; A61K 6/811; A61K 6/813; A61K 6/816

(57) ABSTRACT

A method for manufacturing a ceramic inkjet ink for printing on a dental material substrate includes processing solid particle components to a particle size from 0.8 to 1 m, at a particle size distribution of D98 and adding an effective amount of additives to the solid particle components to produce the ink capable of being dispensed from an inkjet printhead onto a surface of a 3-dimensional dental material substrate having an irregular shape and uneven surfaces, while the 3-dimensional dental material substrate is simultaneously translated and rotated under the inkjet printhead.

### CLAIM OF PRIORITY UNDER 35 U.S.C. § 119

The present application for patent claims priority to Provisional Application No. 63/627,670 entitled “SYSTEM AND METHOD FOR GLAZING DENTAL RESTORATIONS WITH CERAMIC INK VIA INKJET PRINTER” filed Jan. 31, 2024, and assigned to the assignee hereof and hereby expressly incorporated by reference herein.

### BACKGROUND

### Field

Various embodiments relate to ceramic inkjet ink and glaze inkjet compositions for coloring dental material substrates that mimic the coloration and translucency of human teeth.

### Background

Conventional inkjet printing of ceramic inkjet inks onto ceramic products has suffered to a number of technical limitations including stability issues, as well as limited color intensity and color range. Although improvements to this technology are still being sought, greater technical and commercial success has been achieved through the use of solvent based ceramic inkjet ink compositions comprising dispersions of pigments suitable for ceramic decoration. Because non-contact inkjet printing is used, there are few demands for the surface shape of the printing medium. However, the requirements for a ceramic inkjet ink composition can be very challenging because of the small size of the print head nozzles on the inkjet printer, and the higher solids loading of the ceramic inkjet inks.

When pigment particle size is small, color can be more difficult to achieve. Much effort has been expended in recent years to obtain inkjet compositions that could be applied using conventional inkjet equipment to print various substrates while not interfering with the color development of the ceramic inkjet ink.

A primary issue facing ceramic inkjet inks is when they are printed over unfired glazes, as they are prone to producing poor print quality. This poor print quality manifests itself as blurring, variable color strength, gloss differential across the substrate, cracking, and bleed. These print quality issues, which become apparent after firing a glaze and ink combination, have been found to be related to the poor absorption of the inks into the first-down glaze. The inks also must satisfy several criteria. First, they must have the correct rheological and other properties such that they can easily be ejected, or dispensed, from the nozzles of the inkjet printhead. Attempts at printing ceramics by the inkjet process often fail, owing to the pronounced tendency of the specifically heavy and coarse color powders to form sediment, produced when the solid components of the ink settle out from aqueous or alcoholic suspensions and lead to blocking of the print head nozzles of the inkjet printing apparatus.

Another concern in formulating ceramic inkjet inks is that the conventional glazes used to formulate the inks consist of mixtures of raw materials of metal oxides which, in suitable proportions, react together during the firing cycle to achieve fusion of the metal oxides as a homogenous whole. Such formulations can contain an excess of fine grains of the solid metal oxide and pigment particles, which leads to instability and particle aggregation.

Thus, there has been little success in addressing the many demands of a ceramic inkjet ink so that the ceramic inks are capable of meeting all of the physicochemical properties required for inkjet printing (e.g., particle size, particle size distribution, viscosity, density, surface tension, firing heat resistance, etc.). Nor have there been any ceramic inks that are capable of being printed on the uneven, rough, surface of a substrate, where the surface area is very small, while the substrate is simultaneously being translated and/or rotated, in a three dimensional plane during the inkjet printing, and capable of maintaining their desired coloristic properties after the surface of the substrate has been subject to firing.

### SUMMARY

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

Various embodiments provide glaze inkjet and ceramic inkjet ink compositions that can be used to modify the physical and aesthetic properties of the surface of a dental material substrate, and thus provide a functional coating or glaze inkjet and ceramic inkjet ink composition. The physical and aesthetic properties of the substrate can be changed or modified with the compositions and can affect various attributes of the substrate after firing including gloss, contrast gloss (luster), specular reflectance (metallic or glassy appearance), relief, and slipperiness.

An embodiment is directed to a ceramic inkjet ink for dental material substrates. The ink includes a ceramic pigment. The ceramic pigment includes metal oxide solid particles having a submicron particle size of less than 1 micron at a particle size distribution from D98 to D10 and inorganic pigment solid particles having a submicron particle size of less than 1 micron at a particle size distribution from D98 to D10. The ink further includes a solvent and a dispersing agent. The ink has a solids particle loading of about 10 wt. % to about 50 wt. %, wherein the ink exhibits good jet ability upon being uniformly ink jetted on the surface of a dental material substrate by an inkjet printer, and wherein the ink is capable of being fused to the surface of the dental material substrate by firing at elevated temperatures and of maintaining its aesthetic coloristic properties after firing.

Another embodiment is directed to a concentrated glaze paste composition for dental material substrates. The composition includes a glaze powder including a metal oxide solid particle having a submicron particle size of less than 1 micron at a particle size distribution ranging from D98 to D10. The composition further includes a solvent, a dispersing agent and an additive. The concentrated glaze paste composition has a solids particle loading ranging from 10 wt. % to 70 wt. %.

Another embodiment is directed to a method of manufacturing digital ceramic inkjet ink for dental material substrates. The method includes wet milling a concentrated glaze powder in a solvent, in the presence of a first dispersing agent and a first anti-settling agent, to produce a concentrated glaze paste having solid glaze powder particles of a particle size from 0.8 to 1 μm, at a particle size distribution of D98. The method further includes wet milling a colored stain powder in a solvent, in the presence of a dispersing agent and an anti-settling agent, to produce a concentrated stain glaze paste having solid particles of a particle size from 0.8 to 1 μm, at a particle size distribution of D98. The method further includes mixing the concentrated glaze paste with the concentrated stain glaze paste in a mixer mill to produce a stain-glaze mixture, adding a dilution solution comprising a mixture of solvents and additives to the stain-glaze mixture to produce a precursor color ceramic inkjet composition having a solids loading content from about 20 wt. % to about 60 wt. %, based on the total weight of the colored ceramic inkjet ink composition and desired ink jetting properties, and filtering the precursor color ceramic inkjet composition first through a 5 micron pore size filter, and subsequently through a 2 micron pore size filter, to remove any oversized solid particles, thereby obtaining the digital ceramic inkjet ink having a viscosity of 12 to 25 mPa·s at a jetting temperature and jetting conditions.

Another embodiment is directed to a method for manufacturing a ceramic inkjet ink for printing on a dental material substrate includes processing solid particle components to a particle size from 0.8 to 1 μm, at a particle size distribution of D98 and adding an effective amount of additives to the solid particle components to produce the ink capable of being dispensed from an inkjet printhead onto a surface of a 3-dimensional dental material substrate having an irregular shape and uneven surfaces, while the 3-dimensional dental material substrate is simultaneously translated and rotated under the inkjet printhead.

### DETAILED DESCRIPTION

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and circuits have not been described in detail as not to unnecessarily obscure aspects of the present invention.

### Definitions

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

“coefficient of expansion” (or CTE) refers to a physical or material property requirement for the compositions of various embodiments of the present invention to be applied on the surface of a dental material substrate, and the dental material substrate itself, after each has undergone firing. The CTE is indicative of curvature, bending, chips, cracks, etc., for the compositions, or the dental material substrate, and describes how, or the rate at which, the compositions or substrate expands or contracts when subjected to temperature changes. The CTE of the glaze inkjet composition, and the ceramic inkjet ink composition, should be compatible with the CTE of the underlying dental material substrate to prevent cracking, crazing, or deformation during the firing and cooling cycles. As used in the dental industry, CTE typically refers to the CTE of Cerabien ZR (CZR) and is tailored to match the CTE of zirconia frameworks, which typically ranges from 10.0×10⁻⁶/° C. to 10.5×10⁻⁶/° C., depending on the zirconia type used. Mismatched CTE values for the glaze inkjet composition, the ceramic inkjet ink composition, and the underlying dental material substrate can lead to thermal stress, and result in aesthetic, or structural, failure. For example, the CTE for the ceramic inkjet ink composition should match the CTE for a crown dental material that is made of zirconia. A broad CTE range is desired, as it accommodates for variability in the compositions and dental material substrates. As a non-limiting example, the CTE of the glaze inkjet compositions, the ceramic inkjet ink compositions, and the underlying dental material substrates according to various embodiments may range from 65×10⁻⁷/° C. to 75×10⁻⁷/° C., preferably ranges from 50×10⁻⁷/° C. to 80×10⁻⁷/° C., and more preferably ranges from 9.0×10⁻⁶/° C. to 11.0×10⁻⁶/° C.

“surface tension” refers to a physicochemical characteristic that governs the liquid behavior of the ceramic inkjet ink composition, and glaze inkjet composition, of various embodiments, at fluid interfaces. More particularly, it impacts the jetting of the compositions using the printhead nozzles of the inkjet printer, and the interaction of the compositions with the dental material substrate. The surface tension also influences the drop formation, jetting stability, and drop-dental material substrate interaction. The surface tension for the ceramic inkjet ink compositions and glaze inkjet compositions according to various embodiments is tailored for the specific printhead design, inkjet composition, and dental material substrate requirements, and can be further adjusted through the inclusion of additives such as surfactants, polymers, or other additives, to avoid inkjet composition spread and achieve optimal printhead performance. As a non-limiting example, the surface tension for the ceramic inkjet ink compositions and glaze inkjet compositions according to various embodiments may range from 20 mN/m to 40 mN/m, and preferably ranges from 25 mN/m to 30 mN/m.

“sedimentation” refers to a physical property addressing the gravitational settling of solid particles of the glaze inkjet composition and ceramic inkjet ink composition of the present invention over a period of time under standard storage conditions. The phenomenon impacts the stability of glaze inkjet and ceramic inkjet ink compositions and nozzle clogging of the printhead. Sedimentation is influenced by factors including particle size, density, viscosity of the mediums, and interactions between particles of the composition. It can be determined via an optical analysis of a liquid dispersion of the composition and is expressed as a weight percent of the glaze inkjet and ceramic inkjet ink composition. The sedimentation rate for inkjet inks is typically designed to be extremely low or negligible over the ink's shelf life, since it can cause serious issues with printhead performance and print quality. As a non-limiting example, the sedimentation rate for the glaze inkjet and ceramic inkjet ink compositions according to various embodiments may be lower than 0.1 wt. %, and preferably 0.013 wt. % to 0.06 wt. %.

“jetting temperature” refers to the temperature of the glaze inkjet and ceramic inkjet ink composition at the printhead. As a non-limiting example, the jetting temperature according to various embodiments may range from 30° C. to 60° C., and preferably from 35° C. to 45° C.

“particle size” refers to the average particle size for the particles of the glaze inkjet and ceramic inkjet ink composition and may be designed to be very small (e.g., in the submicron or nanoscale range and meeting the commonly accepted definition of a nanoparticle) to ensure smooth jetting and prevent printhead nozzle clogging. The submicron particle size may be achieved by milling the glaze inkjet and ceramic inkjet ink composition to have a particle size small enough to prevent sedimentation and ensure stable suspension of the particles for passing through the printhead nozzle, which typically has a diameter of 15 μm to 50 μm. As a non-limiting example, the particle size for the particles of the glaze inkjet and ceramic inkjet ink composition according to various embodiments may be sub-micron in nature and range from 3 microns to below 1 micron.

“particle size distribution” refers to the volume distributed median particle diameter (i.e., equivalent spherical diameter). For example, the term “D98” refers to the 98^{th} percentile volume based median particle size (i.e., the diameter below which 98% by volume of the particle population is found). So, a particle size of 0.80 μm at a particle size distribution of D50, would refer to the median of the particle size distribution in which 50% of the volume of the control sample would have an equivalent diameter of less than 0.80 μm.

“viscosity” refers to the shear rate for the glaze inkjet and ceramic inkjet ink composition, measurable at the jetting temperature using a rotational viscometer at shear rates of 100 to 1000 (l/s/) or a high-frequency rheometer measuring frequency at 50-500 Hz. The viscosity for the compositions should be low enough to enable them to be printed by an inkjet printer (at room temperature). As a non-limiting example, the viscosity for the compositions ranging from 3 mPa·s to 200 mPa·s, at a shear rate ranging from 100 l/s to 1000 l/s.

“pH” and “conductivity” for the ceramic inkjet ink compositions of the present invention are measured at 25° C. using an Oakton 510 series pH/conductivity meter.

“good jet ability” refers to an adequate drop formation for the glaze inkjet and ceramic inkjet ink composition when ejected from an inkjet printer printhead at different drop volumes. There should be no satellites or drop break up, which can be detrimental to the printed image quality, usually verified by inkjet testing on a drop watcher machine.

“good printed image inkjet quality” refers to the image being compliant with the end use application, and is usually verified using a series of tests such as line straightness, wicking, feathering, dot gain, color shades, etc.

“storage stability” refers to the number of days, weeks, or months, that a ceramic inkjet ink composition can be stored without any significant settling or degradation, which can lead to poor performance.

“open time” refers to the time by which a print head can be left uncapped (hence open) and then when jetting is recommenced, a complete start-up of all nozzles. Normal open times are at least one hour.

“blurring” refers when a printed design generated with intermediate ink discharge due to the rate of ink absorption into the glaze being deficient. It is apparent on the halftone printed areas of the design, where the neighboring ink drops coalesce at the surface, giving a somewhat stained appearance, as if the image was printed at very low resolution with big ink drops.

“bleeding” or “bleed” refers to solid areas in the print design where the non-absorbed ink flows out over the damp glaze leading to distortion of the image edges and also mixing with other areas of the printed resulting in color halos and poor print resolution.

“resolubility” refers to the time taken to resolubilize the ceramic inkjet ink composition which has air dried in a digital print head due to poor maintenance, or downtime. The expectation is that when using a standard flush, cleaning or maintenance liquid, the ceramic inkjet ink composition is resolubilized in less than 10 minutes, thus enabling any blocked nozzles in the printhead to be recovered. Data measured on draw down samples on metal substrates can clearly demonstrate that by including small amounts of a metal or metal oxides in the ceramic inkjet ink compositions, the color shift can be significantly reduced to a level which is acceptable in the inkjet printing industry, namely a ΔE of less than 3.0.

“coverage” refers to a physical property that relates to the viscosity and melt surface tension of the glaze inkjet and ceramic inkjet ink composition, as a characteristic fundamental for achieving continuous covering by the composition on the surface of the dental material substrate. As a non-limiting example, the coverage for the glaze inkjet and ceramic inkjet ink composition after firing may be lower than 200 g/m², preferably lower than 150 g/m², and more preferably lower than 100 g/m², where the sintering and softening temperatures upon firing may range from 700° C. to 1,300° C.

### Ceramic Pigments

According to various embodiments, a glaze inkjet composition may applied directly on surface of a dental material substrate (e.g. crown). The glaze inkjet composition may also applied over wet and dried ceramic inkjet ink composition or ceramic stain ink. The interaction between the dental material substrate, the glaze inkjet composition and the ceramic inkjet ink composition is critical for the final outcome for the inkjet printed on the dental substrate material.

One of the key challenges in formulating glaze inkjet compositions having a high solids loading is achieving good dispersion of the particles and preventing sedimentation and caking during storage while still maintaining a very low viscosity for the composition. Further challenges also involve preventing inkjet printhead channel and nozzle blockage. From the ink jetting perspective, the challenge is reliable jetting small satellite free drop sizes of ink, while jetting at high frequency. Due to the environment where the inkjet printer will be operated, the glaze inkjet composition also requires an improved nozzle open time (i.e., the ability to inkjet the composition without nozzle failure for a prolong period). Accomplishing this requires careful consideration, and a delicate balance, in formulating the glaze inkjet composition and any ceramic inkjet inks formulated from the composition.

Accordingly, various embodiments are directed to a glaze inkjet and ceramic inkjet ink composition having a completely distinct nature that renders them capable of being inkjet applied to a dental material substrate without clogging the print head or spray nozzles. Thus, they preferably have appropriate physical properties, such as viscosity within the range of 5 to 50 mPa·s, a surface tension of from about 20 to 40 mN·m and a density of from about 0.8 to about 1.5 g/ml. Advantageously, the compositions according to various embodiments preferably exhibit little or no volatility, and are stable with the materials used to make inkjet printing systems (e.g. print heads, nozzles, delivery lines, etc.).

An object of the present invention is to develop a ceramic inkjet ink composition that works with the requirements of a ceramic system. For example, they should be capable of generating the desired surface affect (i.e., the desired physical surface change) once applied on the surface of a substrate after firing of the substrate. They should also be capable of interacting with a glazed substrate surface to become part of it. They should also contain a sufficiently high solids loading to allow reasonable productivity and allow for enough resolution despite the high solids loading.

Various embodiments involve developing inkjet formulations employing additives, such as, rheology modifiers, viscosity modifiers, surface tension modifiers, in order to formulate glaze and inkjet ink compositions in a manner where they can easily be incorporated into a base fluid at a let-down stage to improve or adjust their physicochemical properties and substrate interaction behavior.

Various embodiments may involve developing a glaze inkjet and ceramic inkjet ink composition formulation route that employs a short list of additives (e.g., dispersants, rheological modifiers, binders, humectant, surfactants, and other additives) that are fully compatible with the components used to formulate the glaze inkjet and ceramic inkjet ink compositions, and where the additives can easily be added to, for example, a concentrated paste of the glaze, or pigmented stain, to fine-tune the inkjet composition as appropriate, at an end-user location.

Various embodiments may involve developing a glaze powder having particles that can be milled, with an intermedium, to generate submicron particles suspended in solvent that ensure full material compatibility of the glaze inkjet composition and additives (e.g., dispersants, rheological modifiers, binders, humectant, surfactants, and other additives) of the glaze inkjet and ceramic inkjet ink compositions.

Various embodiments may involve developing a system for optimizing the complex rheological properties of a glaze inkjet and ceramic inkjet ink composition to match the specific requirements of an inkjet print head of choice having a customized waveform.

Various embodiments may involve developing and formulating key thinner solutions, additives, rheology modifiers, and intermedium capable of being used to formulate glaze inkjet and ceramic inkjet ink compositions in a manner that they can be easily incorporated into a base fluid at a let-down stage to improve rheology and substrate interaction behavior.

### Ceramic Pigments

The ceramic pigments used to create the ceramic inkjet ink composition according to various embodiments may be represented metal oxides and inorganic pigments. Other types of pigments could also be used and include: organo-metallic pigments, complexes, micronized pigments, colloidal metals, nano-pigments, and reactive sole precursors for in situ pigment synthesis, such as polyol and sol-gel synthesis. The Classification and Chemical Descriptions of the Complex Inorganic Color Pigments, Fourth Edition (2010) contains a list of ceramic pigments.

Examples of suitable metal oxides may be include oxide, silicate, or zirconate compounds of Si, Fe, Co, Sn, Zn, Pr, Ni, or Cr. In particular, suitable metal oxides useful for preparing the glaze forming material of the present invention may include, but are not limited to, silicon dioxide SiO₂, boron oxide B₂O₃, La₂O, B₂C₃, along with oxides of Al (Al₂O₃), R₂O (generically referring to the group of alkaline oxides Li₂O, Na₂O, K₂O, B(BaO), ZnO, ZrO₂, SnO₂, TiO₂, P₂O₅, zinc borate, RO (generically referring to the group of oxides of: MgO, CaO, SrO, BaO, Mg (MgO), or mixtures thereof. There is almost an infinite number of fundamental oxides that can be used to make the glaze forming material. However, most are silicate (Si), zinc oxide (ZnO), and boric acid (H₃BO₃) based, with different concentrations of alkali, copper, and lime. SiO₂ is a preferred oxide since it has the facility to generate insoluble glassy materials when combined with other oxides. Oxides based on SiO₂ have a proportion of between 20 wt. % and 49 wt. %; based on Al₂O₃ have a proportion of between 0.01 wt. % and 1 wt. %; also metal oxide combinations, for example, based on LiO₂+Na₂O+K₂O; based on MgO+CaO+SrO+BaO; based B₂O₃+LiO₂+ZnO; based B₂O₃+LiO₂; based on Na₂O+K₂O+TiO₂; and additional metal oxides known to those skilled in the ceramics industry; where the weight percentage is in relation to the total weight of metal oxides in the glaze inkjet composition.

The metal oxides used in the glaze inkjet composition according to various embodiments may range from 0.1 wt. % to 10 wt. %, preferably 2 wt. % to 5 wt. %, and more preferably 0.1 wt. % to 2 wt. %, based on the weight of the glaze inkjet composition.

The inorganic pigments represented in the ceramic pigment used herein may refer to pigments which are, or are at least partially, the oxides of metals, are heat resistant, and do not completely burn off during the firing process once the ink is applied to the surface of the substrate where the inorganic pigment gets deposited on the surface of the substrate, and where most of its optical properties (e.g., color, optical density, gloss, etc.) are maintained after firing.

Examples of suitable inorganic pigments include, but are not limited to, silica, sands, feldspar, alumina, clay, zirconium silicate, zinc oxide, zinc cadmium sulfide dolomite, borates, calcite, kaolin, quart silica, barium carbonate, wollastonite, tin oxide, nepheline, bismuth oxide, colemanite, calcium carbonate, cerium oxide, cobalt oxide, copper chromite black spinel, cobalt chromite, cobalt titanium spinel, copper oxide, iron oxide, iron oxide red, aluminum phosphate, iron carbonate, manganese oxide, sodium fluoride, chromium oxide, strontium carbonate, lithium carbonate, blue green spinel, cobalt aluminate blue spinel, nickel antimony titanium yellow rutile, titanium dioxide rutile and anatase cadmium sulfide, cadmium suloslenide, spodumene, talc, magnesium oxide, manganese ferrite, cristobalite, rutile, bismuth vanadate anatase, sodium feldspar, vanadium oxide, ammonium pentavanadate, and mixtures thereof. Examples of suitable sole precursor in situ polyol synthesis prepared ceramic pigments, and their color, include, but are not limited to, CoFe₂O4 (a black color), CoAl₂O₄ (a blue color), Ti(Cr, Sb)O2 (a yellow color), Au (a magenta color), and Cu (a magenta color), and mixtures thereof.

Examples of other suitable inorganic pigments which may also be used for preparing the ceramic inkjet ink compositions according to various embodiments include, but are not limited to, bindheimite (Pb₂Sb₂O₇ a yellow color), olivine (Co₂SiO₄ a blue color), spinel (Co, Ni, Fe, Cr, Mn)₃O₄ and CoAl₂O₄ a blue and black color), cassiterite ((Sn, Cr)O₂ a pink color), corundum ((Cr, Al)₂O₃ a green color), uvarovite (Ca₃Cr₂Si₃O₁₂ a green color), malayaite (Ca(Sn, Cr)SiO₅ a burgundy color), greenockite (Cd (S, Se) an orange-red color), rutile (Ti, Cr, Sb, W)O₂ an orange and tobacco color), zircon ((Zr, Pr)SiO₄, (Zr, V)(Si, V)O₄, Fe₂O₃ZrSiO₄, a yellow, turquoise, and red color respectively), baddeleyite ((Zr, V)O₂ a yellow color), cerianite ((Ce, Pr)O₂ a red color), perovskite (Y(Al, Cr)O₂ a red color), cuprorivaite (CaCuSi₄O₁₀ a blue color), caeruleum (a blue color), copper-wollastonite ([Ca,Cu]₃Si₃O₉ a green color), and mixtures thereof.

The inorganic pigment particles should be sufficiently small (e.g., micronized or nano particles) to permit free flow of the ceramic inkjet ink composition, formulated using the colorant particles, through the ejecting nozzles of an inkjet printing device. To accomplish this, the particles may be milled, e.g., wet milled, to have an average sub-micron particle size of less than 1 μm at a particle size distribution ranging from D10 to D98 and adjusted by introducing additives capable of adjusting, or modifying, the resolubility, good jetting, sedimentation speed, coefficient of expansion, surface tension, storage stability, transparency, and color gamut, of the composition.

The inorganic pigments according to various embodiments may be used in the range of 0.1 wt. % to 10 wt. %, preferably 0.01 wt. % to 5 wt. %, and more preferably 0.1 wt. % to 2 wt. %, based on the weight of the ceramic inkjet ink composition.

### Dispersing Agent

In order to incorporate the ceramic pigments into the ceramic inkjet ink composition, the ceramic pigments may be manufactured and stably stored as a concentrate in water or solvent. This is typically achieved by dispersing the pigment materials into water or solvent with a dispersing agent.

Examples of suitable dispersing agents include, but are not limited to, polymeric dispersing resins such as polyvinyl alcohols, polyvinylpyrrolidone, polyacrylic acid, acrylic acid-acrylonitrile copolymers, vinyl acetate-acrylate copolymers, acrylic acid-acrylate copolymers, styrene-acrylic acid copolymers, styrene-methacrylic acid copolymers, styrene-methacrylic acid-acrylate copolymers, styrene-alpha methyl styrene-acrylic acid copolymers, styrene-alpha methyl styrene-acrylic acid-acrylate copolymers, styrene-maleic acid copolymers, styrene-maleic anhydride copolymers, vinyl naphthalene-acrylic acid copolymers, vinyl naphthalene-maleic acid copolymers, vinyl acetate-maleate copolymers, vinyl acetate-crotonic acid copolymers, and vinyl acetate-acrylic acid copolymers, and the salts thereof, among others. Examples of suitable copolymer dispersing resins include, but are not limited to, any form of random copolymer, block copolymer, alternating copolymer and graft copolymer, among others. Examples of suitable dispersing resin salts include, but are not limited to, sodium hydroxide, potassium hydroxide and salts of basic compounds such as ammonia, ethylamine, diethanolamine, triethanolamine, propylamine, isopropyl amine, dipropylamine, butylamine, isobutyl amine, diethanolamine, triethanolamine, triisopropanol amine, dimethyl ethanolamine, amino methyl propanol, and morpholine, among others.

Examples of commercially available dispersing agents include, but are not limited to, Disper BYK 111, 180, 182, 184, 190, 191, 192, 194, 2010, 2015, and 2155 (all available from Altana); Additol XW 6565, XW 6575, XW 330 and XL 6577 (available from Allnex); Efka PU 4009, 4010, Fa4564; Joncryl 67, 678, 8500, 586, 611, 680, 682, 683 and 69 (available from BASF); Solsperse J945, J955, J980, J981, J944, J950 (available from Lubrizol); Anti-terra-250, E-sperse (available from Ethox); Fluijet (available from Lamberti), PVP K-15, K-30, K-60, and Tego 7502 and 752W (available from Evonik); among others. The dispersing agents according to various embodiments may be used alone, or in combination, and may be in a range of 1 wt. % to 20 wt. % based on the weight of the glaze inkjet and ceramic inkjet ink composition.

### Solvent

The glaze inkjet and ceramic inkjet ink compositions according to various embodiments may include organic solvents, along with water as a co-solvent. The organic solvents for use in various embodiments may include hydrophilic, hydrophobic, polar, non-polar, and aqueous solvents. The use of a solvent helps reduces the evaporation rate of any water components, and further preventing drying of the compositions in the nozzles of the inkjet printheads, thereby preventing the nozzles from clogging. The solvents also provide a secondary function of performing as a wetting aid, allowing the inkjet drops to spread on the substrate. Finally, the solvents enhance the solubility of the components of the compositions and facilitate penetration of the compositions into the dental material substrate. The use of water as a co-solvent would cause the compositions not to be highly flammable or volatile, and should not be water containing any ionic impurities, such as an ion exchange or distilled water.

Examples of suitable organic solvents include, but are not limited to, C₁₂ to C₄₀ hydrocarbons, dearomatized hydrocarbons of isoparaffins, paraffinns, and cycloparaffins, traded under the commercial brand D40, D80, and D120 (available from Exxsol), dearomatized aliphatic hydrocarbons, C₁₂ to C₄ o aliphatic solvents, C₁₂ to C₄₀ linear alkanes such as paraffins, ester solvents; glycols such as methyl glycol, ethyl glycol, butyl glycol, alkylene glycol, alkylene glycol ether or ether acetate type, 4-hydroxy-4-methyl-2-pentanone, diethylene glycol butyl ether, diethylene glycol ethyl ether, diethylene glycol methyl ether, diethylene glycol monoethyl ether acetate, diethylene glycol monopropyl ether, dipropylene glycol ethyl ether, dipropylene glycol methyl ether, ethylene glycol butyl ether, ethylene glycol ethyl ether, ethylene glycol isopropyl ether, ethylene glycol methyl ether, ethylene glycol propyl ether, glycerine carbonate, N-methyl 2-pyrrolidone, glycerol, propylene glycol, glycol ethers such as propylene glycol ethyl ether, propylene glycol ethyl ether acetate, propylene glycol methyl ether, propylene glycol n-propyl ether, triethylene glycol butyl ether, triethylene glycol methyl ether, tripropylene glycol, tripropylene glycol methyl ether; alcohols such as methanol, ethanol, propanol, isopropyl alcohol, butanol, polyols, ethoxy propanol, methoxy butanol, ethylene glycol, propylene glycol, dipropylene glycol, glycerine, and poly ethylene glycol, among others), ketones and ketone alcohols such as acetone, diacetone alcohol, diacetone propanol, and methyl ethyl ketone, methyl butyl ketone, cyclohexanone; ethers such as methyl acetate, ethyl acetate, butyl acetate, propyl acetate, methoxy propyl acetate, ethyl-3-ethoxy-propanol, tetrahydrofuran, dioxane, and alkylethers, ethers of polyhydric alcohols, ethylene glycol monomethyl ether, ethylene glycol monoethyl ether, ethylene glycol monoethyl ether acetate, di(ethyleneglycol) monomethyl ether); nitrogen-containing solvents such as 2-pyrrolidone, and N-methyl 2-pyrrolidone; sulfur-containing solvents such as 2,2′-thiodiethanol, dimethylsulfoxide, tetramethylene sulfone, and sulfolane, and sugars and derivatives thereof such as glucose, oxyethylene adducts of glycerin, and oxyethylene adducts of diglycerin propylene glycol and/or dipropylene glycol, N-methyl pyrollidone, and urea, among others.

The organic solvents may be used alone, or in combination, and when used alone are used in a range of 50 wt. % to 80 wt. % based on the weight of the glaze inkjet or ceramic inkjet ink composition. The water-based solvents (i.e., organic solvent with water as co-solvent) use water in a range of 25 wt. % to 60 wt. %, based on the weight of the glaze inkjet or ceramic inkjet ink composition.

### Binder

The ceramic inkjet ink compositions and glaze inkjet compositions according to various embodiments include, but are not limited to, polymeric binders that are compatible with the other components of the composition. The binders impart a number of desired properties, including, but not limited to, substrate adhesion, stability, smear resistance, viscosity, surface tension, coating penetration, optical density, color depth, adhesion, highlighter resistance, resolubility and crust resistance, among others. Examples of suitable binders, and commercially available binders, include, but are not limited to, polyurethane dispersions, polyacrylics and polyurethane dispersions-acrylic co-polymers, hydroxy propyl cellulose, PVP series; Lubrijet N240, Carboset 560, PrintRite DP375 and XPD-3276 (available from Lubrizol); Tego Variplus DS50, Tego Silkopur 8081 (available from Evonik); Joncryl 90, Joncryl 8055, Joncryl 8050-E, Joncryl ECO 2189-E, Joncryl 8211, Joncryl ECO 2177-E (available from BASF); UC-84 from Alberdingk Boley; Daotan TW 6450, Daotan 6490, Reshydrol SF 8000, Reshydrol SF 8010, Reshydrol 8011, Daotan TW 7000, Daotan TW 6425, Daotan VTW 6460, Daotan 7225 (available from Allnex); Optiflo L 1400, Optiflo T 1000 and Optiflo T 1010 (available from Altana); Phenoxy PKHW 34, Phenoxy PKHW 35 and Phenoxy PKHW 38 (available from); Azelis, Aquazol 5 and Aquazol 50 (available from Chempoint); Neocryl BT21, Neocryl XK-12, Neorez R-2005, Neorez R-4000, Neocryl A-1127, Neocryl A-1125, Neopac E-200, Neocryl A-1131, Neocryl D-2101 from DSM; EPS 546 and Dyflex LP 9419 (available from EPS Materials); Hauthane L-2892, Hauthane L-2897, Hauthane HD-4670 and Hauthane L-2183 (available from C. L. Hauthaway and Sons Inc.); Esajet PU77, Esajet 4518, Esajet AC20, Esajet 200, Esajet 4518, Esajet PU 931, Esajet 5913 (available from Lamberti); Takelac WS-4022 and Takelac WS-5000 (available from Mitsui Chemicals); and Syntran 3101 (available from Zschimmer and Schwarz).

The binders may be used alone, or in combination, and may be in a range of 1 wt. % to 10 wt. %, based on the weight of the glaze inkjet and ceramic inkjet ink composition.

### Surfactant

Surfactants, or wetting agents, are used in the pigment dispersion for the inkjet inks of the present invention to lower the interfacial tension of allowing it to spread on a solid surface, and in various embodiments may include anionic, non-anionic, and cationic surfactants. Examples of suitable anionic surfactants include, but are not limited to, alkane sulphonates, alpha-olefin sulphonates, alkyl benzene sulphonates, alkyl naphthalene sulphonates, acyl methyl taurinates, dialkyl sulfosuccinates, alkyl sulfates, sulfurized olefins, polyoxyethylene alkyl ether phosphates, polycarboxylic acids and mono glycerol phosphate, amphoteric surfactants such as alkylpyridinium salts and non-ionic surfactants such as polyoxyethylene alkyl ethers, polyoxyethylene alkyl phenyl ethers, polyoxyethylene alkyl esters, polyoxyethylene alkyl amides, glycerol alkyl esters and sorbitan alkyl esters, among others. Suitable non-anionic surfactants include, but are not limited to, commercially available non-anionic surfactants, such as BYK 345 and 305, among others.

Examples of suitable cationic surfactants include, but are not limited to, commercially available cationic surfactants, such as BYK 300, 306, 345, 3481, and BYK Dynwet 810, among others.

Examples of commercially available surfactants include, but are not limited to, the PLURONIC® series, TETRONIC® series, ICONOL® series and EFKA 1000, 4000, 5000 and 6000 series (available from BASF Corporation, Parsippany, N.J.); ARQUAD® series (available from Akzo Chemical Inc., Chicago, Ill.); TRITON® series (available from Union Carbide Corp., Danbury, Conn.); SURFONIC® series (available from Texaco Chemical Company, Houston, Tex.); ETHOQUAD® series (available from Akzo Chemical Inc., Chicago, Ill.); ARMEEN® series (available from Akzo Chemical Inc., Chicago, Ill.); SURFYNOL® series (available from Air Products and Chemicals, Inc. Allentown, Pa.); and ETHOMEEN® series (available from Akzo Chemical Inc., Chicago, Il.); Tamol series (available from Dow); Solsperse series 27,000, 40,000, 44,000, 46,000 and 47,000 (available from Lubrizol); BYK 300 (cationic), 305 (non-anionic), 306 (cationic), 342, 345 (non-anionic), 348, 349, 378, 3481 (cationic), BYK Dynwet 810 (cationic), and polyether siloxane co-polymers Tego Wet KL 245 (available from Evonik). The surfactants may used alone, or in combination, and may be in a range of 0.1 wt. % to 3 wt. %, based on the weight of the glaze inkjet and ceramic inkjet ink composition.

### Other Additives

The ceramic inkjet ink compositions according to various embodiments can optionally include additional additives, compatible with the other components of the composition, that impart any number of adjustments, or modifications, to the desired rheological, physical, and optical properties for the ceramic inkjet ink composition. Such properties include, but are not limited to, stability, pH modification, smear resistance, preservatives, viscosity, surface tension, penetration, optical density, color depth, adhesion, highlighter resistance, resolubility, defoaming, preservatives, anti-settling, rheology modifiers, and crust resistance. Additives that can be included in the glaze inkjet and ceramic inkjet ink compositions to address the foregoing properties include, but are not limited to, additives directed to stability, pH modification, smear resistance, preservatives, viscosity, surface tension, penetration, optical density, color depth, adhesion, highlighter resistance, resolubility, defoaming, preservatives, anti-settling, rheology modifiers, and crust resistance antioxidants, ultraviolet absorbers, chelating agents, electric conductivity adjusters, oxygen absorbers, anti-curling agents, and fragrances, among others.

For example, in addition to water and solvent, the glaze inkjet and ceramic inkjet ink compositions according to various embodiments may include a humectant to reduce the rate of evaporation of the water and solvent components and prevent the composition from drying out in the nozzles of the inkjet printer printhead, which can occur during periods of latency. Examples of suitable humectants, having high hygroscopicity and water-solubility, include, but are not limited to: polyols (e.g., glycerol, propylene glycol, and ethylene glycol), alcohol ethers (e.g., diethylene glycol, triethylene glycol), lactams (e.g., 2-pyrrolidone, urea compounds such as urea, 1,3-dimethylimidazolidinone), saccharides (e.g., sorbitol), 1,4-cyclohexanedimethanol, 1-methyl-2-piperidone, N-ethylacetamide, 3-amino-1,2-propanediol, ethylene carbonate; butyrolacetone and Liponic EG-1, among others. The humectants may used alone, or in combination, and may be in a range of 0 wt. % to 5 wt. %, based on the weight of the glaze inkjet and ceramic inkjet ink composition.

Examples of suitable pH modifiers, to adjust or buffer the ceramic inkjet ink composition to a desired pH include, but are not limited to, alkali hydroxides, alkali carbonates and bicarbonates, triethylamine, dimethylethanolamine, triethanolamine, mineral acids, hydrochloric acid, and sulfuric acid, among others. The pH modifiers may be used alone, or in combination, and may be in an amount of 3 wt. % to 10 wt. %, based on the weight of the glaze inkjet and ceramic inkjet ink composition.

Examples of suitable anti-settling agents, used to improve the resistance of pigment and other solids in the glaze inkjet and ceramic inkjet ink composition from settling at the expanse of a raised viscosity, include, but are not limited to, BYK-Chemie GmbH agents sold under the trade names BYK-410, BYK-415 and BYK-430, and may be used alone, or in combination with other additive materials such as binders, rheology additives, and other additives such as fumed silica (Aerosil 200). The anti-settling agents may be used alone, or in combination, and may be in an amount of 0 wt. % to 3 wt. %, based on the weight of the glaze inkjet and ceramic inkjet ink composition.

Examples of suitable rheology modifiers, to adjust the rheology of the glaze inkjet and ceramic inkjet ink composition, include, but are not limited to, BYK 7420, polyvinyl pyrrolidones such as PVP K-30 and PVP K-90, among others. The rheology modifiers may be used alone, or in combination, in an amount of 0 wt. % to 2 wt. %, based on the weight of the glaze inkjet and ceramic inkjet ink composition.

Examples of suitable penetrating agents, to reduce bleeding of the ceramic inkjet ink composition when applied to a substrate, include, but are not limited to, alkyl alcohols having 1 to 4 carbon atoms (e.g., ethanol), glycol ethers (e.g., ethylene glycol monomethyl ether), diols (e.g., 1,2-alkyl diols), formamide, acetamide, dimethylsulfoxide, sorbitol and sulfolane, among others. The penetrating agents may be used alone, or in combination, and may be in a range of 1 wt. % to 5 wt. %, based on the weight of the glaze inkjet and ceramic inkjet ink composition.

Examples of suitable defoamers to inhibit the formation of foam include, but are not limited to, silicone-based or non-silicone defoamers. Commercially available defoamers include, but are not limited to, DC62, DC65, DC 68, DC71 and DC74 (available from Dow Corning); TegoAirex® 900, 910, 916, 920, 931, 936, 940, 944, 945, 950, 962, 980, 986, 901W, 902W, 904W, and TEGO FOAMEX N, FOAMEX 1488, 1495, 3062, 7447, 800, 8030, 805, 8050, 810, 815N, 822, 825, 830, 831, 835, 840, 842, 843, 845, 855, 860, and 883, TEGO FOAMEX K3, TEGO FOAMEX K7/K8 and TEGO TWIN 4000 (available from Evonik Industries/Tega); Tergitol® L-61, L-62, L-64 and L-101 (available from Dow Chemical); BYK-066N, 088, 055, 057, 1790, 020, BYK-A 530, 067A, and BYK 354 (available from BYK); Agitan 120, 150, 160, 271, 290, 298, 299, 350, 351, 731, 760, 761, and 777 (available from Munzing); and Surfynol 104PA, AD01, DF-110, DF-58, DF-62, DF-66, DF-695, DF-70, and MD-20 (available from Air Products), among others. The defoamers may be used alone, or in combination, and may be in a range of 0.1 wt. % to 3 wt. % based on the weight of the glaze inkjet and ceramic inkjet ink composition.

Examples of suitable preservatives, such as biocides and fungicides, to inhibit the growth of microorganisms in the ceramic inkjet ink compositions, include, but are not limited to, sodium benzoate, pentachlorophenol sodium, 2-pyridinethiol-1-oxide sodium, sodium sorbate, sodium dehydroacetate, benzisothiazolinone, 1,2-dibenzothiazolin-3-one, 1-(3-chlorallyl)-3,5,7-triaza-1 azoniaadamantane chloride (CTAC), methylisothiazolinone, and chloromethylisothiazolinone, among others. Commercially available biocides include UCARCIDE® 250 (available from Union Carbide Company), Proxel® CRL, Proxel® BDN, Proxel® GXL, Proxel® XL-2, Proxel® TN (available from Arch Chemicals), Dowicil® (available from Dow Chemical), Nuosept® (available from Huls America, Inc.), Omidines® (available from Olin Corp.), Nopcocides® (available from Henkel Corp.), Troysans® (available from Troy Chemical Corp.), bromo-nitro-propane-diol, isothiazolinone, ethylenedioxydimethanol, or iodo-propynyl butyl carbamate (which are all marketed under the trade name Intercide, available from Akcros Chemicals), Nipacide (available from Clariant), sodium dehydroacetate (Geogard 111S, available from Lonza), sodium benzoate (Vancide 51, available from R. T. Vanderbilt), sodium pyridinethiol-1-oxide (Sodium Omadine, available from Arch Chemicals), sodium salt of o-phenylphenol (Dowicide A, available from DOW Chemical), ethyl p-hydroxybenzoate (Nipastat Sodium, available from Aako), and XBINX® (available from PMC Specialties Group, Inc.), among others. The preservatives may be used alone, or in combination, and may be in a range of less that 1 wt. %, based on the weight of the glaze inkjet and ceramic inkjet ink composition.

### Glaze

Glazes according to various embodiments may be a composition of vitreous materials that are capable of promoting the fusibility of the glaze inkjet and ceramic inkjet ink composition of the present invention. The glaze is typically derived from a glaze powder that is formed by the addition of solvent, dispersing agents, and optionally additives, to the glaze powder to form a concentrated glaze paste. The concentrated glaze paste include metal oxides of ceramic pigments that have been fused together by grinding in a high shear mill to form a homogeneous whole, in suitable proportions, and then reacted together by firing in a kiln at an elevated temperature and for a sufficient time. In various embodiments, the kiln firing time may range from 30 minutes to 3 hours, and the elevated temperature may range from 800° C. to 1200° C., and may preferably be 850° C. After firing, the metal oxides form a glassy matrix of insoluble flakes or granules. The purpose of the fusion is to render any soluble and/or toxic components insoluble by causing them to combine with metal oxides, such as silicates. The glassy matrix is re-ground, or granulated, into a fine dust or glaze powder, which is also known in the ceramics industry as a “frit” such as, for example, CERABIEN® ZR (available from Kuraray America, Inc., NY, NY). The ceramic metal oxides of the concentrated glaze paste or glaze inkjet composition may be completely sintered by the firing, in such a way to gain suitable esthetic, have fluid proof qualities, have little or no porosity, and become generally resistant to abrasion and chemical attack.

The glaze inkjet composition according to various embodiments may also incorporate water as a co-solvent. The water can reduce the toxicity of certain oxides used, such as PbO, to create the glaze powder. The glaze should be capable of binding to the surface of a dental material substrate, capable of providing coverage for the entire surface of the dental material substrate, and capable of becoming fused to the dental material substrate upon firing at an elevated temperature for a sufficient time. The firing transforms the dental material substrate to a strong and continuous structure. The glaze is typically obtained by rapidly quenching the melted micronized ceramic pigments of the glassy matrix. Conventionally, the firing cycle time for the glassy matrix would last for up to 24 hours or more. However, using a quick-firing kiln, the firing time can be greatly reduced to 25 to 60 minutes. The use of metal oxide ceramic pigments, and firing them, leads to optimal homogeneity, and a quick reaction of the metal oxides of the glaze.

The metal oxide particles of the concentrated glaze paste or glaze inkjet composition, according to various embodiments, should be sufficiently small (e.g., micronized) to permit free flow of the glaze inkjet composition and ceramic inkjet ink composition formulated from the glaze inkjet composition, through the ejecting nozzles of an inkjet printing device. To accomplish this, the metal oxide ceramic pigments may be milled, e.g., wet milled, to an average particle size of less than 1 micron, and preferably to an average particle size ranging from less than 5 microns to 0.05 microns, and more preferably to an average particle size ranging from less than 1 micron to 0.05 microns. The micronized, or nanoparticle, size for the metal oxide ceramic pigments is accompanied by a particle size distribution ranging from D10 to D98. To establish and maintain the small particle size and particle size distribution for the metal oxide ceramic pigments, a dispersing agent, along with other additives, may be added to the ceramic inkjet ink composition and glaze inkjet composition that is capable of adjusting, or modifying, the resolubility, good jetting, sedimentation, coefficient of expansion, surface tension, storage stability, transparency, and color gamut, of these compositions.

### Ceramic Inkjet Ink Composition

The ceramic inkjet ink compositions according to various embodiments may be formulated, for example, by first preparing a precursor clear glaze inkjet composition, which may be formed from a glaze forming material, which may be prepared from a glaze powder such as CERABIEN® ZR (available from Kuraray America, Inc., NY NY), that is wet milled, at high shear, in the presence of a milling medium, in a solvent, and a dispersing agent. Upon wet milling the glazed powder, a clear concentrated glaze paste, typically having a solids loading, or content, of from 40 wt. % to 70 wt. %, is produced. Next, a color stain pigment, such as a color stain made from CERABIEN® ZR (available from Kuraray America, Inc., NY, NY) may then be combined with the clear concentrated glaze paste to achieve a colored glaze paste mixture.

The combined color paste mixture may then be wet milled, at high shear, in the presence of a milling medium, and the pigment particle size and particle size distribution of the combined color paste mixture is monitored and measured periodically, while the mixture is undergoing wet milling, until the target pigment particle size and particle size distribution are reached. Various embodiments achieve many of the goals stated herein through the pigments of the colored glaze paste mixture being well dispersed in the solvent and possessing nano-scale dimensions (e.g., a pigment particle size below 1 μm) for the resulting ceramic inkjet ink composition, formulated from the colored glaze paste mixture, to flow through the small nozzles of the printhead of the inkjet printer. Once the target particle size and particle size distribution are reached, wet milling of the combined color paste mixture is discontinued, and a concentrated color stain paste is obtained. The nano-scale particle size pigments are usually obtained by the wet milling process.

Next in some embodiments, a dilution solution of solvent, surfactant, and a dispersing agent, may be mixed with the concentrated color stain paste, at ambient temperature, to produce a ceramic inkjet ink composition according to various embodiments which can be applied on the surface of a dental material substrate.

Additives may optionally be added to the combined color paste mixture before or while the paste mixture is dispersed, such as a dispersing agent or anti-settling agent, as deemed necessary to achieving the desired physical and optical properties for the ceramic inkjet ink composition and glaze inkjet composition, as these properties are subject to change once these compositions are deposited on the surface of the dental material substrate and exposed to firing. The additives are added in an amount ranging from 1 wt. % to 20 wt. %. The firing process itself requires a certain combination of temperature and time, as well as temperature lowering requirements that may need to be addressed, for example, by the addition of additives. This is because developing of ceramic inkjet ink compositions having lowered firing requirements that maintain a high chemical stability is extremely difficult to obtain, and a compromise typically has to be made between the firing time and temperature, along with the other properties for the compositions.

### Dental Material Substrate

The ceramic inkjet ink compositions according to various embodiments may be formulated for inkjet printing on dental material substrates, such as dental models, dental restorations, occlusal guards, clear aligners, surgical guides, monolithic full dentures, partial dentures, bridges, crowns and artificial teeth. These dental material substrates may manufactured through milling a block of raw substrate or by using a three-dimensional printer (3D printer) that prints a resin, or a nano ceramic hybrid resin, to form substrates that simulate gums, the biome of the oral cavity, and teeth. As the resins are to be used in the manufacture of dental materials for this specific end use, it is important that the resins be classified as a high-end (Class-1) biocompatible resins, meeting the requirements for the biological evaluation of medical devices as specified by the Food and Drug Administration (FDA) under ISO 10993-1. The chemical nature of these high-end biocompatible resins includes ceramic crown polymers, leucite glass ceramics, natural dentin, and lithium disiliate, among others. Examples of commercial resins meeting the biocompatible requirements established by the FDA, include, but are not limited to, resins such as hydroxypropyl cellulose marketed and sold under the tradename Klucel E., Onyx by Sprint Ray; IC-131 by Glidwell; FreePrint Temp UV by Detax; Water Washable Model Resin by Phrozen; Acetomax; Dental SG Resin by Formlabs; Smile Guard Resin Proto 3000 by DeskTop Health; Flexcure; Prime Print; Xdent Model Ortho Dental Resin; and Plonext Removable Die Model Resin.

### Print Testing the Glaze Inkjet and Ceramic Inkjet Ink Compositions

Example glaze inkjet and ceramic inkjet ink compositions according to various embodiments were printed using either a Dimatix DMP2800 printer, or industrial printing presses equipped with either Kyocera KJ4B or Konica Minolta 1024 print heads. All of the examples were printed onto a dental material substrate and evaluated visually. The printed dental material substrate was printed in two different ways.

According to the first way (i.e., Print Protocol 1): As a First Stage, staining was performed by ink jetting an image on a dental material substrate crown, using a glaze inkjet composition according to an embodiment, followed by drying the composition. As a Second stage, glazing was performed, requiring ink jetting on the top of a dental material substrate retoration, several layers of a glaze inkjet composition according to an embodiment, to achieve a required glaze thickness and drying of each layer during the printing (at each step) using an infrared dryer. The glaze on the dental material substrate crown was then subjected to fusing on the dental material crown at an elevated temperature in an oven.

According to the second way (i.e., Print Protocol 2): As a First Stage, pre-glazing was performed by ink jetting a small layer of glaze on a dental material substrate crown, using a glaze inkjet composition (glaze) according to an embodiment, followed by drying the composition during the printing (at each step) using an infrared dryer. As a Second stage, staining was performed by ink jetting an image on a dental material substrate crown, using a glaze inkjet composition according to an embodiment, followed by drying the composition. As a Third Stage, glazing was performed by ink jetting a glaze on the top of a dental material substrate crown, using a glaze inkjet composition according to an embodiment, followed by drying the composition during the printing (at each step) using an infrared dryer. The glaze on the dental material substrate crown was then subjected to fusing on the dental material crown at an elevated temperature in an oven. The printed substrate examples were deemed to pass if they exhibited good jet ability and print image properties. i.e., excellent line straightness, good uniform color density, no edge bleeding, and minimal dot gain. All of the printed substrate examples passed and exhibited good jet ability and print image properties.

### EXAMPLES

The following examples illustrate specific embodiments of the present invention and are not intended, nor should they be interpreted to, limit the scope of the invention thereof in any respect and should not be so construed.

### Example 1

### Milling Medium

In a stirred tank, 95 g of a di propylene glycol methyl ether solvent was slowly added, while stirring to 5 g of a polyvinyl pyrrolidone (PVP30) binder resin at ambient temperature until the mixture was fully dissolved in the solvent. The fully dissolved mixture formed 100 g of a wet milling medium having a 5 vol. % resin content.

### Example 2

### Concentrated Glaze Paste

In stirred tank or vessel, 250 g of the wet milling medium prepared in Example 1, 119 g of di propylene glycol methyl ether solvent, 111 g of a (solvent free) dispersing agent (Disperbyk), 600 g of CERABIEN® ZR (glaze powder available from Kuraray America, Inc., NY, NY), and 200 g of a hydrophilic fumed (silica) anti-settling agent (Aerosil 200), were added together to generate a mixture. The mixture was fully mixed and transferred to wet milling station and wet milled at high shear at ambient temperature. The particle size of the mixture particles was measured every 6 hours until the particles of the mixture maintained a D98 particle size distribution and particles size around 0.8 to 1 microns (i.e., the D98 value particle size Z-average was measured using a Malvern Zetasizer Nano-ZS). Once the desired particle size was achieved, wet milling of the mixture was discontinued and 1000 g of a concentrated glaze paste was obtained.

A characterization of the concentrated glaze paste showed: a 45 wt. % total solids content; a 3.0 vol. % additives content; a 1.3 vol. % resin content; and a 0.10 vol. % content of other components. The physical properties for the concentrated glazed paste were also measured and provided a viscosity of 43-110 mPa·s at 30° C. and a 400 shear rate and 35-85 mPa·s at 30° C. and a 1000 shear rate.

### Example 3

### Concentrated Brown Stain Glaze Paste

In stirred tank or vessel, 20 g of the wet milling medium prepared in Example 1, 229 g of di propylene glycol methyl ether solvent, and 100 g of a (solvent free) dispersing agent (Disperbyk) were added together to generate a mixture. Next, 650 g of a brown stain powder pigment made from CERABIEN© ZR (a glaze powder available from Kuraray America, Inc., NY, NY) and 1 g of hydrophilic fumed silica anti settling agent (Aerosil 200) were added, and the resulting mixture was fully mixed and transferred to a wet milling station and wet milled at high shear at an ambient temperature. The particle size for particles of the mixture was measured every 6 hours until the particles of the mixture maintained a D98 particle size distribution and a particle size of around 0.8 to 1 microns (i.e., the D98 value particle size Z-average was measured using a Malvern Zetasizer Nano-ZS). Once the desired particle size was achieved, wet milling of the mixture was discontinued, and 1000 g of a concentrated brown stain paste was obtained.

A characterization of the concentrated brown stain glaze paste showed: a 63 wt. % total solids content; a 10.0 vol. % additives content; a 0.1 vol. % resin content; and a 0.1 vol. % content of other components. The physical properties for the concentrated brown stain paste were also measured and provided a viscosity of 120-140 mPa·s at 30° C. and a 400 shear rate and 110-120 mPa·s at 30° C. and a 1000 shear rate.

### Example 4

### Concentrated Blue Stain Glaze Paste

In stirred tank or vessel, 20 g of the wet milling medium prepared in Example 1, 230 g of di propylene glycol methyl ether solvent, and 100 g of a (solvent free) dispersing agent (Disperbyk) were added together to generate a mixture. Next, 650 g of a blue stain powder pigment made from CERABIEN® ZR (a glaze powder available from Kuraray America, Inc., NY, NY) was added, and the resulting mixture was fully mixed and transferred to a wet milling station and wet milled at high shear at an ambient temperature. The particle size for particles of the mixture was measured every 3 hours until the particles maintained a D98 particle size distribution and a particle size of around 0.8 to 1 microns (i.e., the D98 value particle size Z-average was measured using a Malvern Zetasizer Nano-ZS). Once the desired particle size was achieved, wet milling of the mixture was discontinued, and 1000 g of a concentrated blue stain paste was obtained.

A characterization of the concentrated blue stain glaze paste showed: a 64 wt. % total solids content; a 10 vol. % additives content; a 0.1 vol. % resin content, and a 0 vol. % content of other ingredients. The physical properties for the concentrated blue stain glaze paste were also measured and provided a viscosity of 140-150 mPa·s at 30° C. and a 400 shear rate and 120-140 mPa·s at 30° C. and a 1000 shear rate.

### Example 5

### Concentrated Yellow Stain Glaze Paste

In stirred tank or vessel, 20 g of the wet milling medium prepared in Example 1, 230 g of di propylene glycol methyl ether solvent, and 100 g of a solvent free dispersing agent (Disperbyk) were added together to generate a mixture. Next, 650 g of a yellow stain powder pigment made from CERABIEN® ZR (a glaze powder available from Kuraray America, Inc., NY, NY) was added, and the resulting mixture was fully mixed and transferred to a wet milling station and wet milled at high shear at an ambient temperature. The particle size for particles of the mixture was measured every 3 hours until the particles maintained a D98 particle size distribution and a particle size of around 0.6 to 0.8 microns (i.e., the D98 value particle size Z-average was measured using a Malvern Zetasizer Nano-ZS). Once the desired particle size was achieved, wet milling of the mixture was discontinued, and 1000 g of a concentrated yellow stain paste was obtained.

A characterization of the concentrated yellow stain paste showed: a 63 wt. % total solids content; a 10 vol. % additives content; and a 0.1 vol. % resin content; and a 0 vol. % content of other components. The physical properties for the concentrated yellow stain paste were also measured and provided a viscosity of 70-80 mPa·s at 30° C. and a 400 shear rate and 65-75 mPa·s at 30° C. and a 1000 shear rate.

### Example 6

### Glaze Inkjet Composition

In stirred tank or vessel, a dilution solution of 384.5 g of di propylene glycol methyl ether solvent and 15 g of a propylene glycol methyl ether solvent were added to 0.5 g of a surfactant (BYK 342) and 30 g of a (solvent free) dispersing agent (Disperbyk 111). The dilution solution was combined with 570 g of the concentrated glaze paste prepared in Example 2, and the resulting mixture was fully mixed at ambient temperature to obtain 1000 g of a glaze inkjet composition, which was filtered to remove any oversized particles using a 5 micron filter, and later using a 2 micron filter.

A characterization of the glaze inkjet composition showed: a 25.7 wt. % total solids content; a 4.71 vol. % additives content; a 0.7 vol. % resin content; a 0.05 vol. % surfactant content; a 23.1 vol. % co-solvent content; and 0.06 vol. % content of other components. The physical properties of the glazed inkjet composition were measured to give a surface tension of 27.6 mNm at 30° C.; a viscosity of 15-25 mPa·s at and a 400 shear rate and 16-23 mPa·s at 30° C. and a 1000 shear rate; a density of 1130 kg/m at 25° C.

### Example 7

### Brown Stain Glaze Ceramic Inkjet Ink Composition

In a high shear mixer mill, 150 g of the concentrated glaze paste made from CERABIEN® ZR (a glaze powder available from Kuraray America, Inc., NY, NY) prepared in Example 2 was combined with 400 g of the concentrated brown stain glaze paste made from CERABIEN® ZR (a glaze powder available from Kuraray America, Inc., NY, NY) prepared in Example 3 to provide a concentrated mixture. Next, the concentration of the concentrated mixture was adjusted to provide a total pigment to concentrated brown stain glaze paste ratio of 2.5. A dilution solution of 434.5 g of di propylene glycol methyl ether solvent, 15 g of a propylene glycol methyl ether solvent, and 0.5 g of a surfactant (BYK 342) was then combined with the concentrated glaze paste and concentrated brown stain glaze paste in a high shear mixer mill. The resulting mixture was then fully mixed at ambient temperature to obtain 1000 g of a brown stain glaze ceramic inkjet ink composition, which was filtered to remove any oversized particles using a 5 micron filter, and later using a 2 micron filter.

A characterization of the brown stain glaze ceramic inkjet ink composition showed: a 31.8% wt. solids content; a 9.0 vol. % glaze content; a 26 vol. % brown stain content; a 4.45 vol. % additives content; a 0.2 vol. % resin content; a 0.05 vol. % surfactant content; a 1.5 vol. % co-solvent content, and 0.06 vol. % content of other components. The physical properties of the brown stain glaze ceramic inkjet ink composition was measured to give a surface tension of 27.7 mNm at 30° C.; a viscosity of 14-17 mPa·s at 30° C. and a 400 shear rate and 13-15 mPa·s at 30° C. and a 1000 shear rate; a density of 1210 kg/m at 25°.

### Example 8

### Blue Stain Glaze Ceramic Inkjet Ink Composition

In a high shear mixer mill, 150 g of a concentrated glaze paste made from CERABIEN® ZR (a glaze powder available from Kuraray America, Inc., NY, NY) prepared in Example 2 was combined with 400 g of the concentrated blue stain glaze paste made from CERABIEN® ZR (a glaze powder available from Kuraray America, Inc., NY, NY) prepared in Example 4 to provide a concentrated mixture. The concentration of the concentrated mixture was adjusted to provide a total pigment to concentrated blue stain glaze paste ratio of 2.5. A dilution solution of 434.5 g of di propylene glycol methyl ether solvent, 15 g of a propylene glycol methyl ether solvent and 0.5 g of a surfactant (BYK 342) was then combined with the concentrated glaze paste and concentrated blue stain glaze paste in a high shear mixer mill. The resulting mixture was then fully mixed at an ambient temperature to obtain 1000 g of a blue stain glaze inkjet ink composition, which was filtered to remove any oversized particles using a 5 micron filter, and later using a 2 micron filter.

A characterization of the blue stain glaze ceramic inkjet ink composition showed: a 32.7 wt. % solids content; a 9.0 vol. % glaze content; a 26 vol. % blue stain content; a 4.45 vol. % additives content; a 0.2 vol. % resin content; a 0.05 vol. % surfactant content; a 1.5 vol. % co-solvent content, and 0.02 vol. % content of other components. The physical properties of the blue stain glaze ceramic inkjet ink composition was measured to give a surface tension of 29.4 mNm at 30° C.; a viscosity of 13-15 mPa·s at 30° C. and a 400 shear rate and 14-16 mPa·s at 30° C. and a 1000 shear rate; a density of 1260 kg/m at 25° C.

### Example 9

### Yellow Stain Glaze Ceramic Inkjet Ink Composition

In a high shear mixer mill, 150 g of a concentrated glaze paste made from CERABIEN® ZR (a glaze powder available from Kuraray America, Inc., NY, NY) prepared in Example 2 was combined with 400 g of the concentrated yellow stain glaze paste made from CERABIEN® ZR (a glaze powder available from Kuraray America, Inc., NY, NY) prepared in Example 5 to provide a concentrated mixture. The concentration of the concentrated mixture was adjusted to provide a total pigment to concentrated yellow stain glaze paste ratio of 2.5. A dilution solution of 434.5 g of di propylene glycol methyl ether solvent, 15 g of a propylene glycol methyl ether solvent, and 0.5 g of a surfactant (BYK 342) was then combined with the concentrated glaze paste and the concentrated yellow stain glaze paste in a high shear mixer mill. The resulting mixture was then fully mixed at an ambient temperature to obtain 1000 g of a yellow stain glaze ceramic inkjet ink composition, which was filtered to remove any oversized particles using a 5 micron filter, and later using a 2 micron filter.

A characterization of the yellow stain glaze ceramic inkjet ink showed: a 31.8 wt. % solids content; a 9.0 vol. % glaze content; a 26 vol. % yellow stain content; a 4.45 vol. % additives content; a 0.2 vol. % resin content; a 0.05 vol. % surfactant content; a 1.5 vol. % co-solvent content, and 0.02 vol. % content of other components. The physical properties of the yellow stain glaze ceramic inkjet ink composition was measured to give a surface tension of 29.9 mNm at 30° C.; a viscosity of 12-15 mPa·s at 30° C. and a 400 shear rate and 14-16 mPa·s at 30° C. and a 1000 shear rate; a density of 1260 kg/m at 25° C.

The previous description of the disclosed embodiments is provided to enable any person skilled in the art to make or use the present invention. Various modifications to these embodiments will be readily apparent to those skilled in the art, and the generic principles defined herein may be applied to other embodiments without departing from the spirit or scope of the invention. Thus, the present invention is not intended to be limited to the embodiments shown herein but is to be accorded the widest scope consistent with the principles and novel features disclosed herein.

What is claimed is:

1. A ceramic inkjet ink for dental material substrates comprising:
a ceramic pigment comprising:
metal oxide solid particles having a submicron particle size of less than 1 micron at a particle size distribution from D98 to D10; and
inorganic pigment solid particles having a submicron particle size of less than 1 micron at a particle size distribution from D98 to D10;
a solvent; and
a dispersing agent;
wherein the ink has a solids particle loading of about 10 wt. % to about 50 wt. %, wherein the ink exhibits good jet ability upon being uniformly ink jetted on the surface of a dental material substrate by an inkjet printer, and wherein the ink is capable of being fused to the surface of the dental material substrate by firing at elevated temperatures and of maintaining its aesthetic coloristic properties after firing.

2. The ceramic inkjet ink of claim 1, wherein the metal oxide solid particle has a submicron particle size from about 0.85 μm to about 0.93 μm at a particle size distribution of D98.

3. The ceramic inkjet ink of claim 1, wherein the metal oxide solid particle has a submicron particle size from about 0.6 μm to about 0.66 μm at a particle size distribution of D90.

4. The ceramic inkjet ink of claim 1, wherein the metal oxide solid particle has a submicron particle size from about 0.39 μm to about 0.4 μm at a particle size distribution of D50.

5. The ceramic inkjet ink of claim 1, wherein the metal oxide solid particle has a submicron particle size from about 0.2 μm to about 0.24 μm at a particle size distribution of D10.

6. The ceramic inkjet ink of claim 1, wherein the amount of metal oxide solid particles is from about 1 wt. % to about 10 wt. %, based on the weight of the ceramic inkjet ink.

7. The ceramic inkjet ink of claim 1, wherein the inorganic pigment solid particle is selected from chromium oxide, titanium dioxide, and iron oxide, or a mixture thereof.

8. The ceramic inkjet ink of claim 1, wherein the inorganic pigment solid particle has a submicron particle size from about 0.85 μm to about 0.93 μm at a particle size distribution of D98.

9. The ceramic inkjet ink of claim 1, wherein the inorganic pigment solid particle has a submicron particle size from about 0.6 μm to about 0.66 μm at a particle size distribution of D90.

10. The ceramic inkjet ink of claim 1, wherein the inorganic pigment solid particle has a submicron particle size from about 0.39 μm to about 0.4 μm at a particle size distribution of D50.

11. The ceramic inkjet ink of claim 1, wherein the inorganic pigment solid particle has a submicron particle size from about 0.2 μm to about 0.24 μm at a particle size distribution of D10.

12. The ceramic inkjet ink of claim 1, wherein the amount of inorganic pigment solid particles is from about 1 wt. % to about 10 wt. %, based on the weight of the ceramic inkjet ink.

13. The ceramic inkjet ink of claim 1, wherein the solvent is selected from polar, non-polar, hydrophobic, hydrophylic, and aqueous solvents, or a mixture thereof.

14. The ceramic inkjet ink of claim 13, wherein the solvent is selected from C₁₂ to C₄₀ hydrocarbons, dearomatized hydrocarbons of isoparaffins, paraffinns, and cycloparaffins, traded under the commercial brand D40, D80, and D120 (available from Exxsol), dearomatized aliphatic hydrocarbons, C₁₂ to C₄₀ aliphatic solvents, C₁₂ to C₄₀ linear alkanes such as paraffins, ester solvents; glycols such as methyl glycol, ethyl glycol, butyl glycol, alkylene glycol, alkylene glycol ether or ether acetate type, 4-hydroxy-4-methyl-2-pentanone, diethylene glycol butyl ether, diethylene glycol ethyl ether, diethylene glycol methyl ether, diethylene glycol monoethyl ether acetate, diethylene glycol monopropyl ether, dipropylene glycol ethyl ether, dipropylene glycol methyl ether, ethylene glycol butyl ether, ethylene glycol ethyl ether, ethylene glycol isopropyl ether, ethylene glycol methyl ether, ethylene glycol propyl ether, glycerine carbonate, N-methyl 2-pyrrolidone, glycerol, propylene glycol, glycol ethers such as propylene glycol ethyl ether, propylene glycol ethyl ether acetate, propylene glycol methyl ether, propylene glycol n-propyl ether, triethylene glycol butyl ether, triethylene glycol methyl ether, tripropylene glycol, tripropylene glycol methyl ether; alcohols such as methanol, ethanol, propanol, isopropyl alcohol, butanol, polyols, ethoxy propanol, methoxy butanol, ethylene glycol, propylene glycol, dipropylene glycol, glycerine, and poly ethylene glycol, among others), ketones and ketone alcohols such as acetone, diacetone alcohol, diacetone propanol, and methyl ethyl ketone, methyl butyl ketone, cyclohexanone; ethers such as methyl acetate, ethyl acetate, butyl acetate, propyl acetate, methoxy propyl acetate, ethyl-3-ethoxy-propanol, tetrahydrofuran, dioxane, and alkylethers, ethers of polyhydric alcohols, ethylene glycol monomethyl ether, ethylene glycol monoethyl ether, ethylene glycol monoethyl ether acetate, di(ethyleneglycol) monomethyl ether); nitrogen-containing solvents such as 2-pyrrolidone, and N-methyl 2-pyrrolidone; sulfur-containing solvents such as 2,2′-thiodiethanol, dimethylsulfoxide, tetramethylene sulfone, and sulfolane, and sugars and derivatives thereof such as glucose, oxyethylene adducts of glycerin, and oxyethylene adducts of diglycerin propylene glycol and/or dipropylene glycol, N-methyl pyrollidone, and urea, and mixtures thereof.

15. The ceramic inkjet ink of claim 1, wherein the amount of solvent is from about 30 wt. % to about 80 wt. %, based on the weight of the ceramic inkjet ink.

16. The ceramic inkjet ink of claim 1, wherein the dispersing agent is selected from polymeric dispersing resins such as polyvinyl alcohols, polyvinylpyrrolidone, polyacrylic acid, acrylic acid-acrylonitrile copolymers, vinyl acetate-acrylate copolymers, acrylic acid-acrylate copolymers, styrene-acrylic acid copolymers, styrene-methacrylic acid copolymers, styrene-methacrylic acid-acrylate copolymers, styrene-alpha methyl styrene-acrylic acid copolymers, styrene-alpha methyl styrene-acrylic acid-acrylate copolymers, styrene-maleic acid copolymers, styrene-maleic anhydride copolymers, vinyl naphthalene-acrylic acid copolymers, vinyl naphthalene-maleic acid copolymers, vinyl acetate-maleate copolymers, vinyl acetate-crotonic acid copolymers, and vinyl acetate-acrylic acid copolymers, and the salts thereof, among others. Examples of suitable copolymer dispersing resins include, but are not limited to, any form of random copolymer, block copolymer, alternating copolymer and graft copolymer, among others. Examples of suitable dispersing resin salts include, but are not limited to, sodium hydroxide, potassium hydroxide and salts of basic compounds such as ammonia, ethylamine, diethanolamine, triethanolamine, propylamine, isopropyl amine, dipropylamine, butylamine, isobutyl amine, diethanolamine, triethanolamine, triisopropanol amine, dimethyl ethanolamine, amino methyl propanol, and morpholine, and mixtures thereof.

17. The ceramic inkjet ink of claim 1, wherein the amount of dispersing agent is from about 1 wt. % to about 20 wt. %, based on the weight of the ceramic ink jet ink.

18. The ceramic inkjet ink of claim 1, further comprising an additive selected from a stability additive, a pH modification additive, a smear resistance additive, a preservative, a viscosity modifying additive, a surface tension additive, a penetration additive, an adhesion additive, a resolubility additive, a defoaming additive, a preservative, an anti-settling additive, a rheology modifying additive, a chelating agent, an oxygen absorber, and mixtures thereof.

19. The ceramic inkjet ink of claim 1, wherein the amount of the additive is from 0 wt. % to about 3 wt. %, based on the weight of the ceramic inkjet ink.

20. The ceramic inkjet ink of claim 1, further comprising a surfactant selected from alkane sulphonates, alpha-olefin sulphonates, alkyl benzene sulphonates, alkyl naphthalene sulphonates, acyl methyl taurinates, dialkyl sulfosuccinates, alkyl sulfates, sulfurized olefins, polyoxyethylene alkyl ether phosphates, polycarboxylic acids, mono glycerol phosphate, amphoteric surfactants, alkylpyridinium salts, polyoxyethylene alkyl ethers, polyoxyethylene alkyl phenyl ethers, polyoxyethylene alkyl esters, polyoxyethylene alkyl amides, glycerol alkyl esters, sorbitan alkyl esters, and mixtures thereof.

21. The ceramic inkjet ink of claim 1, wherein the amount of the surfactant is from about 0.1 wt. % to about 3 wt. %, based on the volume of the ceramic inkjet ink.

22. The ceramic inkjet ink of claim 1, having a viscosity ranging from about 12 mPa·s to about 25 mPa·s at a shear rate ranging from 400 to 1000 s⁻¹ at the jetting temperature.

23. The ceramic inkjet ink of claim 1, having a viscosity from about 12 mPa·s to about 25 mPa·s at a shear rate of 400 s⁻¹ at the jetting temperature.

24. The ceramic inkjet ink of claim 1, having a viscosity from about 12 mPa·s to about 25 mPa·s at a shear rate of 1000 s⁻¹ at the jetting temperature.

25. The ceramic inkjet ink of claim 1, having a surface tension at 30° C. from about 20 mN/m to about 35 mN/m.

26. The ceramic inkjet ink of claim 1, having a density at 25° C. from about 1050 kg/m³ to about 1300 kg/m³.

27. The ceramic inkjet ink of claim 1, further comprising an elasticity of less than 10%.

28. The ceramic inkjet ink of claim 1, wherein the ink maintains its aesthetic coloristic properties after firing at temperatures from 800° C. to 1300° C.

29. A concentrated glaze paste composition for dental material substrates comprising:
a glaze powder comprising a metal oxide solid particle having a submicron particle size of less than 1 micron at a particle size distribution ranging from D98 to D10;
a solvent;
a dispersing agent; and
an additive;
wherein the concentrated glaze paste composition has a solids particle loading ranging from 10 wt. % to 70 wt. %.

30. The concentrated glaze paste composition of claim 29, wherein the metal oxide solid particle has a submicron particle size from about 0.85 μm to about 0.93 μm at a particle size distribution of D98.

31. The concentrated glaze paste composition of claim 29, wherein the metal oxide solid particle has a submicron particle size from about 0.6 μm to about 0.66 μm at a particle size distribution of D90.

32. The concentrated glaze paste composition of claim 29, wherein the metal oxide solid particle has a submicron particle size from about 0.39 μm to about 0.4 μm at a particle size distribution of D50.

33. The concentrated glaze paste composition of claim 29, wherein the metal oxide solid particle has a submicron particle size from about 0.2 μm to about 0.24 μm at a particle size distribution of D10.

34. The concentrated glaze paste composition of claim 29, wherein the amount of the metal oxide solid particles is from about 1 wt. % to about 10 wt. %, based on the weight of the concentrated glaze paste.

35. The concentrated glaze paste composition of claim 29, wherein the solvent is selected from polar, non-polar, hydrophobic, hydrophylic, and aqueous solvents, or a mixture thereof.

36. The concentrated glaze paste composition of claim 35, wherein the solvent is selected from is selected from C₁₂ to C₄₀ hydrocarbons, dearomatized hydrocarbons of isoparaffins, paraffinns, and cycloparaffins, traded under the commercial brand D40, D80, and D120 (available from Exxsol), dearomatized aliphatic hydrocarbons, C₁₂ to C₄₀ aliphatic solvents, C₁₂ to C₄₀ linear alkanes such as paraffins, ester solvents; glycols such as methyl glycol, ethyl glycol, butyl glycol, alkylene glycol, alkylene glycol ether or ether acetate type, 4-hydroxy-4-methyl-2-pentanone, diethylene glycol butyl ether, diethylene glycol ethyl ether, diethylene glycol methyl ether, diethylene glycol monoethyl ether acetate, diethylene glycol monopropyl ether, dipropylene glycol ethyl ether, dipropylene glycol methyl ether, ethylene glycol butyl ether, ethylene glycol ethyl ether, ethylene glycol isopropyl ether, ethylene glycol methyl ether, ethylene glycol propyl ether, glycerine carbonate, N-methyl 2-pyrrolidone, glycerol, propylene glycol, glycol ethers such as propylene glycol ethyl ether, propylene glycol ethyl ether acetate, propylene glycol methyl ether, propylene glycol n-propyl ether, triethylene glycol butyl ether, triethylene glycol methyl ether, tripropylene glycol, tripropylene glycol methyl ether; alcohols such as methanol, ethanol, propanol, isopropyl alcohol, butanol, polyols, ethoxy propanol, methoxy butanol, ethylene glycol, propylene glycol, dipropylene glycol, glycerine, and poly ethylene glycol, among others), ketones and ketone alcohols such as acetone, diacetone alcohol, diacetone propanol, and methyl ethyl ketone, methyl butyl ketone, cyclohexanone; ethers such as methyl acetate, ethyl acetate, butyl acetate, propyl acetate, methoxy propyl acetate, ethyl-3-ethoxy-propanol, tetrahydrofuran, dioxane, and alkylethers, ethers of polyhydric alcohols, ethylene glycol monomethyl ether, ethylene glycol monoethyl ether, ethylene glycol monoethyl ether acetate, di(ethyleneglycol) monomethyl ether); nitrogen-containing solvents such as 2-pyrrolidone, and N-methyl 2-pyrrolidone; sulfur-containing solvents such as 2,2′-thiodiethanol, dimethylsulfoxide, tetramethylene sulfone, and sulfolane, and sugars and derivatives thereof such as glucose, oxyethylene adducts of glycerin, and oxyethylene adducts of diglycerin propylene glycol and/or dipropylene glycol, N-methyl pyrollidone, and urea, and mixtures thereof.

37. The concentrated glaze paste composition of claim 29, wherein the amount of the solvent is from about 30 wt. % to about 80 wt. %, based on the weight of the concentrated glaze paste.

38. The concentrated glaze paste composition of claim 29, wherein the additive is selected from a stability additive, a pH modification additive, a smear resistance additive, a preservative, a viscosity modifying additive, a surface tension additive, a penetration additive, an adhesion additive, a resolubility additive, a defoaming additive, a preservative, an anti-settling additive, a rheology modifying additive, a chelating agent, an oxygen absorber, and mixtures thereof.

39. The concentrated glaze paste composition of claim 29, wherein the amount of the additive is from 0 to about 3 wt. %, based on the weight of the concentrated glaze paste.

40. The concentrated glaze paste composition of claim 29, further comprising a glaze concentrate having a viscosity ranging from about 43 mPa·s to about 150 mPa·s at a shear rate ranging from 400 to 1000.

41. The concentrated glaze paste composition of claim 29, further comprising a glaze concentrate having a viscosity from about 43 mPa·s to about 150 mPa·s at a shear rate of 400.

42. The concentrated glaze paste composition of claim 29, further comprising a glaze concentrate having a viscosity from about 35 mPa·s to about 140 mPa·s. at a shear rate of 1000.

43. The concentrated glaze paste composition of claim 29, wherein the composition has an elasticity of less than 10%.

44. A method of manufacturing digital ceramic inkjet ink for dental material substrates, the method comprising:
wet milling a concentrated glaze powder in a first solvent, in the presence of a first dispersing agent and a first anti-settling agent, to produce a concentrated glaze paste having solid glaze powder particles of a particle size from 0.8 to 1 m, at a particle size distribution of D98;
wet milling a colored stain powder in a second solvent, in the presence of a second dispersing agent and a second anti-settling agent, to produce a concentrated stain glaze paste having solid particles of a particle size from 0.8 to 1 μm, at a particle size distribution of D98;
mixing the concentrated glaze paste with the concentrated stain glaze paste in a mixer mill to produce a stain-glaze mixture;
adding a dilution solution comprising a mixture of solvents and additives to the stain-glaze mixture to produce a precursor color ceramic inkjet composition having a solids loading content from about 20 wt. % to about 60 wt. %, based on the total weight of the colored ceramic inkjet ink composition and desired ink jetting properties; and
filtering the precursor color ceramic inkjet composition first through a 5 micron pore size filter, and subsequently through a 2 micron pore size filter, to remove any oversized solid particles, thereby obtaining the digital ceramic inkjet ink having a viscosity of 12 to 25 mPa·s at a jetting temperature and jetting conditions.

45. The method of claim 44, wherein the first solvent and second solvent are the same, the first dispersing agent and the second dispersing agent are the same, and the first anti-settling agent and the second anti-settling agent are the same.

46. The method of claim 44, wherein:
wet milling a concentrated glaze powder in a solvent comprises wet milling the glaze powder in a plurality of solvents,
the wet milling medium used in the wet milling chamber is selected from zirconia, silicon nitrite, silicon carbide, and mixtures thereof, and
the digital ceramic inkjet ink has a solids loading content ranging from about 20 vol. % to about 60 wt. %, based on the total weight of the digital ceramic inkjet ink, and solids particle size from 0.8 m to 1 μm, at a particle size distribution of D98.

47. The method of claim 44, wherein:
the concentrated glaze stain paste comprises:
about 45 wt. % to about 85 wt. % metal oxide and inorganic pigment;
about 2 wt. % to about 20 wt. % dispersing agent; and
about 10 wt. % to 55 wt. % solvent,
wherein the concentrated glaze stain paste is wet milled in the presence of the dispersing agent and solvents in a wet milling chamber containing zirconia, silicon nitrite, or silicon carbide milling medium.

48. A method for manufacturing a ceramic inkjet ink for printing on a dental material substrate comprising,
processing solid particle components to a particle size from 0.8 to 1 μm, at a particle size distribution of D98; and
adding an effective amount of additives to the solid particle components to produce the ink capable of being dispensed from an inkjet printhead onto a surface of a 3-dimensional dental material substrate having an irregular shape and uneven surfaces, while the 3-dimensional dental material substrate is simultaneously translated and rotated under the inkjet printhead.
